(19) United States
(12) Patent Application Publication
(10) Pub. No.: US 2017/0261495 A1
(43) Pub. Date: Sep. 14, 2017

(54) MODEL SYSTEMS FOR SCREENING MODULATORS OF mTOR SIGNALING

(71) Applicant: SHRINERS HOSPITALS FOR CHILDREN, Tampa, FL (US)

(72) Inventors: Peter B. CRINO, Moorestown, NJ (US); Jack A. PARENT, Ann Arbor, MI (US)

(73) Assignee: SHRINERS HOSPITALS FOR CHILDREN, Tampa, FL (US)

(21) Appl. No.: 15/605,206

(22) Filed: May 25, 2017

Related U.S. Application Data
(63) Continuation of application No. PCT/US2015/062623, filed on Nov. 25, 2015.

(51) Int. Cl. G01N 33/50; C12N 5/0793; C12N 5/074

(52) U.S. Cl. CPC G01N 33/5058; C12N 5/0696; C12N 5/0619; G01N 33/502; G01N 33/5029; G01N 2800/2857

(57) ABSTRACT

The presently disclosed subject matter relates to the generation of induced pluripotent stem cell (iPSC)-derived neuronal cell lines from subjects diagnosed with polyhydramnios-megalencephaly-symptomatic-epilepsy (PMSE) and assays making use of such cell lines to identify mammalian target of rapamycin (mTOR) signalling modulators as well as anti-epileptogenic compounds.

### 1. CROSS-REFERENCE TO RELATED APPLICATIONS

This application is a continuation of International Patent Application No. PCT/US2015/062623, filed Nov. 25, 2015, which claims priority to U.S. Provisional Patent Application Ser. No. 62/084,427, filed Nov. 25, 2014, the contents of which are incorporated by reference in their entireties, and to each of which priority is claimed.

### 2. INTRODUCTION

The presently disclosed subject matter relates to the generation of induced pluripotent stem cell (iPSC)-derived neuronal cell lines from subjects diagnosed with polyhydramnios-megalencephaly-symptomatic-epilepsy (PMSE) and assays making use of such cell lines to identify mammalian target of rapamycin (mTOR) signalling modulators as well as anti-epileptogenic compounds.

### 3. BACKGROUND OF THE INVENTION

The identification of hyperactive mTOR signaling as a cause of neurodevelopmental disorders associated with severe epilepsy and cognitive disability provides a major conceptual breakthrough for therapeutic approach and development. Reducing mTOR signaling with targeted inhibitors such as rapamycin (called sirolimus in clinical parlance) provides a new mechanistic therapeutic approach for epilepsy and neurobehavioral deficits. While clinical trials with sirolimus or everolimus show clear reduction in the size of renal and brain tumors in tuberous sclerosis complex (TSC) (Krueger et al., 2010), the effects on epilepsy are minimal in TSC and there are no documented effects cognition or behavior, e.g., autism. A critical limitation in relying on clinical trials to further investigate efficacy of mTOR inhibitors for epilepsy, cognition, and behavior therapy in mTOR-associated disorders is the often heterogeneous and complex interplay between genotype (e.g., large deletion, small nonsense, or missense), structural alterations in brain architecture, and the lack of a genotype-phenotype correlation. Thus, before mTOR cascade manipulation can be effectively used in clinical studies with more complex and heterogeneous disorders, a model system is necessary that is reliable, reproducible, and that can be tested in a relatively directed fashion.

### 4. SUMMARY OF THE INVENTION

The presently disclosed subject matter relates to iPSC-derived neuronal cell lines from subjects diagnosed with PMSE. PMSE results from a recessive mutation in STE20-related adaptor protein alpha (STRADA) gene. The recessive mutation can be a homozygous deletion of exons 9-13 of the STRADA gene. In certain embodiments, the neuronal cell lines exhibit aberrant mammalian target of rapamycin (mTOR) activation. In certain embodiments, the neuronal cell lines exhibit enhanced mTOR activation. In certain embodiments, the neuronal cell lines exhibit neurite outgrowth defects, cell motility defects, and/or neuronal hyperexcitability. Neuronal hyperexcitability is associated with epilepsy.

The presently disclosed subject matter also provides assays for identifying mTOR signalling modulators. In certain embodiments, these assays include providing an iPSC-derived neuronal cell line from a subject diagnosed with PMSE; determining initial intrinsic excitability of the neuronal cell line; contacting the neuronal cell line with a test compound; and determining resulting intrinsic excitability of the neuronal cell line contacted with the test compound. In certain embodiments, a reduction in the resulting intrinsic excitability in comparison to the initial intrinsic excitability indicates that the test compound is an mTOR signalling inhibitor. In certain embodiments, an increase in the resulting intrinsic excitability in comparison to the initial intrinsic excitability indicates that the test compound is an mTOR signalling agonist. In certain embodiments, the initial intrinsic excitability is determined by measuring the intrinsic excitability with a whole-cell current-clamp recording device. In certain embodiments, the neuronal cell line is contacted with the test compound for from about 3 days to about 7 days.

In certain embodiments, the assays for identifying an mTOR signalling modulator include providing an iPSC-derived neuronal cell line from a subject diagnosed with PMSE; measuring initial neurite outgrowth of the neuronal cell line; contacting the neuronal cell line with a test compound; and determining resulting neurite outgrowth of the neuronal cell line contacted with the test compound. In certain embodiments, a change, e.g., a defect, in the neurite outgrowth in the presence of the test compound in comparison to the neurite outgrowth in the absence of the test compound indicates that the test compound is an mTOR signalling modulator. In certain embodiments, the neuronal cell line is contacted with the test compound for from about 3 days to about 7 days.

In certain embodiments, the assays for identifying an mTOR signalling modulator include providing an iPSC-derived neuronal cell line from a subject diagnosed with PMSE; measuring initial cell motility of the neuronal cell line; contacting the neuronal cell line with a test compound; and determining resulting cell motility of the neuronal cell line contacted with the test compound. In certain embodiments, a change, e.g., a defect, in the cell motility in the presence of the test compound in comparison to the cell motility in the absence of the test compound indicates that the test compound is an mTOR signalling modulator. In certain embodiments, the neuronal cell line is contacted with the test compound for about 1 hour.

In certain embodiments, the assays for identifying an mTOR signalling inhibitor include providing an iPSC-derived neuronal cell line from a subject diagnosed with PMSE; measuring initial phosphorylation status of at least one mTOR substrate of the neuronal cell line; contacting the neuronal cell line with a test compound; and determining resulting phosphorylation status of the at least one mTOR substrate of the neuronal cell line contacted with the test compound. In certain embodiments, a reduction in the phosphorylation status of the at least one mTOR substrate in the presence of the test compound in comparison to the phosphorylation status of the at least one mTOR substrate in the absence of the test compound indicates that the test compound is an mTOR signalling inhibitor. In certain embodiments, the at least mTOR substrate is selected from the group consisting of ribosomal S6 protein, death-associated protein 1 (DAP1), and Autophagy-related protein 13 (ATG13). In certain embodiments, measuring the initial and resulting phosphorylation status is by a method selected from the group consisting of Western blot and immunohistochemistry.

In certain embodiments, the presently disclosed subject matter provides assays for identifying an anti-epileptogenic compound. In certain embodiments, the assays include providing an iPSC-derived neuronal cell line from a subject diagnosed with PMSE; determining initial intrinsic excitability of the neuronal cell line; contacting the neuronal cell line with a test compound; and determining resulting intrinsic excitability of the neuronal cell line contacted with the test compound. In certain embodiments, a reduction in the resulting intrinsic excitability in comparison to the initial intrinsic excitability indicates that the test compound is an anti-epileptogenic compound. In certain embodiments, the intrinsic excitability is determined by measuring the intrinsic excitability with a whole-cell current-clamp recording device. In certain embodiments, the neuronal cell line is contacted with the test compound for from about 3 days to about 7 days.

In certain embodiments, the assays for identifying an anti-epileptogenic compound include providing an iPSC-derived neuronal cell line from a subject diagnosed with PMSE; measuring initial neurite outgrowth of the neuronal cell line; contacting the neuronal cell line with a test compound; and determining resulting neurite outgrowth of the neuronal cell line contacted with the test compound. In certain embodiments, a change, e.g., a defect, in the neurite outgrowth in the presence of the test compound in comparison to the neurite outgrowth in the absence of the test compound indicates that the test compound is an anti-epileptogenic compound. In certain embodiments, the neuronal cell line is contacted with the test compound for from about 3 days to about 7 days.

In certain embodiments, the assays for identifying an anti-epileptogenic compound include providing an iPSC-derived neuronal cell line from a subject diagnosed with PMSE; measuring initial cell motility of the neuronal cell line; contacting the neuronal cell line with a test compound; and determining resulting cell motility of the neuronal cell line contacted with the test compound. In certain embodiments, a change, e.g., a defect, in the cell motility in the presence of the test compound in comparison to the cell motility in the absence of the test compound indicates that the test compound is an anti-epileptogenic compound. In certain embodiments, the neuronal cell line is contacted with the test compound for about 1 hour.

In certain embodiments, the assays for identifying an anti-epileptogenic compound include providing an iPSC-derived neuronal cell line from a subject diagnosed with PMSE; measuring initial phosphorylation status of at least one mTOR substrate of the neuronal cell line; contacting the neuronal cell line with a test compound; and determining resulting phosphorylation status of the at least one mTOR substrate of the neuronal cell line contacted with the test compound. In certain embodiments, a reduction in the phosphorylation status of the at least one mTOR substrate in the presence of the test compound in comparison to the phosphorylation status of the at least one mTOR substrate in the absence of the test compound indicates that the test compound is an anti-epileptogenic compound. In certain embodiments, the at least mTOR substrate is selected from the group consisting of ribosomal S6 protein, death-associated protein 1 (DAP1), and Autophagy-related protein 13 (ATG13). In certain embodiments, measuring the phosphorylation status is by a method selected from the group consisting of Western blot and immunohistochemistry.

In certain embodiments, the assays for identifying an anti-epileptogenic compound include providing an iPSC-derived neuronal cell line from a subject diagnosed with PMSE; measuring initial spontaneous action potentials of the neuronal cell line; contacting the neuronal cell line with a test compound; and determining spontaneous action potentials of the neuronal cell line contacted with the test compound. In certain embodiments, a reduction in the spontaneous action potentials in the presence of the test compound in comparison to the spontaneous action potentials in the absence of the test compound indicates that the test compound is an anti-epileptogenic compound. In certain embodiments, measuring the spontaneous action potentials is by an electrophysiological technique. In certain embodiments, the electrophysiological technique is a patch clamp recording. In certain embodiments, the assays for identifying an anti-epileptogenic compound include providing an iPSC-derived neuronal cell line from a subject diagnosed with PMSE; measuring initial abnormal spiking of the neuronal cell line; contacting the neuronal cell line with a test compound; and determining resulting abnormal spiking of the neuronal cell line contacted with the test compound. In certain embodiments, a reduction in the abnormal spiking in the presence of the test compound in comparison to the abnormal spiking in the absence of the test compound indicates that the test compound is an anti-epileptogenic compound. In certain embodiments, measuring the initial and resulting abnormal spiking is by an electrophysiological technique. In certain embodiments, the electrophysiological technique is a patch clamp recording.

### 5. DETAILED DESCRIPTION OF THE INVENTION

The presently disclosed subject matter relates to neuronal cell lines derived from a cell, e.g., a fibroblast, isolated from a subject diagnosed with PMSE and assays for identifying certain molecules, e.g., mTOR inhibitors and anti-epileptogenic compounds, using these cell lines.

5.1. Definitions

Unless otherwise defined, all technical and scientific terms used herein have the same meanings as commonly understood by one of ordinary skill in the art to which the presently disclosed subject matter belongs. Although methods and materials similar or equivalent to those described herein can be used in the practice or testing of the presently disclosed subject matter, suitable methods and materials are described below.

An “individual,” “patient” or “subject,” as used interchangeably herein, can be a human or non-human animal. Non-limiting examples of non-human animal subjects include non-human primates, dogs, cats, mice, rats, guinea pigs, rabbits, pigs, fowl, horses, cows, goats, sheep and cetaceans.

“An anti-epileptogenic compound” refers to a compound that either prevents or delays the onset of epilepsy, if given prior to epilepsy onset, or that can alleviate seizure severity, prevent or reduce epilepsy progression or pharmaco-responsiveness if given after epilepsy onset.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to +/−20%, preferably up to +/−10%, more preferably up to +/−5%, and more preferably still up to +/−1% of a given value. Alternatively, particularly with respect to biological systems or processes, the term can mean within an order of magnitude, preferably within 5-fold, and more preferably within 2-fold, of a value.

5.2. Generation of iPSC-Derived Neurons and Neural Progenitor Cells

The presently disclosed subject matter provides neuronal cell lines derived from a fibroblast isolated from a subject diagnosed with PMSE. Also provided herein are assays to define links between mTOR activation and human epilepsy. The presently disclosed neuronal cell lines can provide the basis for high-throughput pre-clinical platforms to test the effects of mTOR inhibitors on neuronal morphology and function, as well as to build a pipeline for Phase I clinical trials for epilepsy, cognitive disability, and autism. The presently disclosed neuronal cell lines can be used as a cell assay reagent to test anti-epileptogenic compounds. For example, there are no AKT3, MTOR, PI3K, PTEN, TSC1, or TSC2 null human neuronal cell lines, and thus, there is a clear need for a human cell platform to study mTOR dysregulation in epilepsy. The presently disclosed neuronal cell lines thus can provide screening assays for therapy development. Furthermore, the presently disclosed neuronal cell lines can provide substrates for testing a variety of mTOR pathway inhibitors. The presently disclosed neuronal cell lines can be used to investigate and define electrophysiological responses and the effects of mTOR pathway modulators that can be tested prior to differentiation or after differentiation, permitting assessment of whether preventative therapies could block the development of epilepsy. The presently disclosed neuronal cell lines can provide ideal platforms to define new strategies to treat both seizures and behavioral deficits in mTOR-associated disorders. In addition, there are evolving areas in which mTOR signaling has been implicated such as spinal cord injury (Lu et al., 2012), hypoxia-ischemia (Chen et al., 2012), and Alzheimer's disease (Gouras, 2013) in which the presently disclosed neuronal cell lines that exhibit constitutive mTOR activation prove invaluable for assaying drug effects on for example, neurite outgrowth and cell signaling.

PMSE (OMIM# 611087) is a rare recessive neurodevelopmental disorder found in Old Order Mennonite communities (Puffenberger et al., 2007) characterized by intractable epilepsy, craniofacial dysmorphism, severe neurocognitive and behavioral deficits, and a high (38%) childhood mortality rate. PMSE results from a homozygous deletion of exons 9-13 of the STE20-related adaptor a gene (STRADA) on human chromosome 17 (17q23.3), which encodes the STRADA protein. STRADα normally binds and exports the protein kinase serine/threonine kinase 11 (STK11; also known as LKB1) out of the nucleus, where they bind to M025 to form a trimeric complex that has an inhibitory effect on mammalian target of rapamycin (mTOR) signaling through the sequential phosphorylation of AMP kinase (AMPK) and the tuberous sclerosis complex 1/tuberous sclerosis complex 2 (TSC1/TSC2) complex. Thus, STRADA protein serves as an upstream AMPK regulator and an mTOR signaling inhibitor. Due to a socio-cultural Founder effect limiting marriage to within the Mennonite community, all PMSE patients share an identical genotype (a 5 exon deletion in STRADA). Neuroimaging and pathological analysis of PMSE brains have revealed subcortical heterotopia, and regions of focal dysplasia.

PMSE brain specimens, lymphocytes, and fibroblasts show enhanced mTOR activation (Puffenberger et al., 2007; Orlova et al., 2010; Parker et al., 2013). For example, Osborne reported loss of STRADA leads to activation of signaling by the mTOR complex 1 (mTORC1), a multiprotein complex that contains mTOR and regulatory associated protein of mTOR (raptor), because the trimeric STRADA/LKB1/M025 complex no longer exerts its inhibitory effects on this pathway (Osborne, 2010). It was found that abnormalities in morphology, motility, and path finding resulting from STRADA loss depend on aberrant mTOR activation as changes can be rescued with rapamycin (Orlova et al., 2010; Parker et al., 2013). In a small clinical trial involving five PMSE patients, treatment with sirolimus prior to or at the onset of seizures suppressed the development of intractable epilepsy (Parker et al., 2013). These results are distinct from those in TSC and may reflect the fact that PMSE patients include a clinically and genetically homogeneous population, and that the effect results from the direct action of sirolimus on neurons, all of which lack functional STRADA. Thus, PMSE provides an ideal disorder to investigate aberrant neuronal mTOR signaling because, unlike TSC, FCD, or HME, the pathophysiology results from a single mutation affecting all neurons in a uniform fashion.

The neuronal cell lines of the presently disclosed subject matter are derived from a cell, e.g., a fibroblast, isolated from a subject diagnosed with PSME. Subjects with PMSE share the identical STRADA deletion, and thereby lacking STRADA protein expression (Puffenberger et al., 2007; Orlova et al., 2010; Parker et al., 2013). Thus, no genotypic variability is expected in PMSE subjects, and there is a 100% genotype-phenotype correlation (Puffenberger et al., 2007). Subjects with PMSE represent a homogeneous cell population for study using pharmacological, cell biological, and electrophysiological approaches. The PMSE subject can be a human subject, and can be a female or a male subject. In certain embodiments, the PMSE subjects are aged between 1 year and 5 years. PMSE subjects meet clinical criteria for PMSE, e.g., macrocephaly, facial dysmorphism, joint laxity.

The neuronal cell lines of the presently disclosed subject matter can be generated by using Induced Pluripotent Stem Cells (iPSC) reprogramming of cells, e.g., fibroblasts, obtained from subjects diagnosed with PMSE. In general, iPSC are somatic cell-derived cell lines that can be reprogrammed back into an embryonic-like pluripotent state that enables the development of an unlimited source of any type of human cell needed for therapeutic purposes. For example, iPSC can be prodded into becoming beta islet cells to treat diabetes, blood cells to create new blood free of cancer cells for a leukemia patient, or neurons to treat neurological disorders. In 2006, Takahashi et al., established for the first time murine embryonic stem (ES)-like cell lines from mouse embryonic fibroblasts (MEFs) and skin fibroblasts by simply expressing four transcription factor genes encoding Oct4, Sox2, Klf4, and c-Myc (Takahashi et al., 2006). iPSC exhibit similar morphology and growth properties as ES cells and express ES cell-specific genes. Transplantation of iPSC into immunodeficient mice can result in the formation of germ-cell-tumor (teratoma)-containing tissues from all three germ layers, confirming the pluripotent potential of iPS cells. iPSC can be generated from different cell types, such as neuronal progenitor cells, keratinocytes, hepatocytes, B cells, and fibroblasts of mouse tail tips, kidneys, muscles, and adrenal glands. For successful reprogramming, differentiation status, which itself may depend on the epigenetic genomic state of a somatic cell, must be reset to a pluripotent state. Patient-specific iPSC can provide unprecedented opportunities to elucidate disease mechanisms in vitro, to carry out drug screening and toxicology studies, and to advance cell replacement therapy in regenerative medicine (Colman & Dreesen 2009). Reprogramming of fibroblasts from patients with Mendelian and complex genetic disorders—such as amyotrophic lateral sclerosis, type 1 diabetes, Parkinson's disease, and Duchenne muscular dystrophy—allows the establishment of disease-specific iPSC. To study the disease mechanism, one of the key issues is whether the affected cell type derived from iPSC can recapitulate the disease phenotype.

In accordance with the presently disclosed subject matter, iPSC are derived from cells, e.g., fibroblasts, obtained from a subject diagnosed with PMSE, and the PMSE iPSC can be reprogrammed to become a PSME neuronal progenitor cell or a PSME neural cell. For reprogramming, the PMSE iPSC can be electroporated with episomal plasmid DNA for Sox2/Klf4, Oct3/4, 1-Myc and GFP using an electroporator and plated in iPSC medium. After about one week, the PMSE iPSC can be plated onto mouse embryonic fibroblasts (MEFs) feeders. PMSE iPSC can be characterized for expression of pluripotency genes, differentiation into three germ layers, and karyotype. PMSE iPSC can be differentiated into neurons using, e.g., a dual SMAD inhibitor protocol (Shi et al., 2012).

5.3. Assays of Identifying mTOR Inhibitors

The PMSE-derived neuronal cell lines of the presently disclosed subject matter exhibit aberrant mTOR activation. In certain embodiments, the presently disclosed neuronal cell lines exhibit enhanced mTOR activation. In certain embodiments, the presently disclosed neuronal cell lines exhibit neurite outgrowth defect. In certain embodiments, the presently disclosed neuronal cell lines exhibit cell motility defect. mTOR activation can be investigated using a variety of assays in PMSE-derived neurons and neural progenitor cells prior to differentiation. Suitable assays include, but are not limited to, assays relating to neuronal morphology and neurite outgrowth, and assays relating to cell motility/migration.

Assays relating to neuronal morphology and neurite outgrowth can be used to determine mTOR activation in the presently disclosed neuronal cell lines. A neuron typically consists of two morphological structures, the round neuronal cell body (called “soma”) and the elongated neuronal protrusions (called “neurites”). To determine the efficacy of a particular pharmacological perturbation on neuronal regeneration using high-content screening techniques, automatic quantification of several morphological features is necessary. These features include, but are not limited to, soma number, soma size, neurite length, and neurite branching complexity. There are many tools capable of quantifying neurite morphology, such as NeuronIQ (Xu et al., 2006), NeuronMetrics (Narro et al., 2007), NeuronJ (Meijering et al., 2004), NeuronStudio (Wearne et al., 2005), NeuriteIQ (Zhang et al., 2007), NeuriteTracer (Pool et al., 2008), and NeuronCyto (Yu et al., 2009) for 2D applications; FARSIGHT (Bjornsson et al., 2008), Neuromantic (Myatt et al., 2008), Neuron_Morpho (Brown et al., 2005), and V3D (Peng et al., 2010) for 3D applications. In certain embodiments, neurite outgrowth of the presently disclosed neuronal cell lines is measured.

Assays relating to cell motility/migration can also be used to determine mTOR activation in the presently disclosed neuronal cell lines. In certain embodiments, a modified version of the in vitro wound healing “scratch assay” developed in fibroblasts (Parker et al., 2013) is used to quantify the cell motility in the presently disclosed neuronal cell lines. The basic steps of an in vitro scratch assay include creating a “scratch” in a cell monolayer, capturing the images at the beginning and at regular intervals during cell migration to close the scratch, and comparing the images to quantify the migration rate of the cells (Liang et al., 2007). In certain embodiments, images are taken about every 5 minutes for about 20 hours to follow the directional course of the cell front as well as individual cells.

Furthermore, the PMSE-derived neuronal cell lines of the presently disclosed subject matter exhibit neurophysiological abnormalities. In certain embodiments, the presently disclosed neuronal cell lines exhibit neuronal hyperexcitability. Altered neural excitability can disrupt information processing in neural circuits and can predispose neurons to synchronous activation; this phenotype provides a strong candidate cellular mechanism underlying both the intellectual disability and epilepsy that are pervasive in PMSE-derived neurons, and other disorders associated with hyperactive mTORC1 signaling. Neuronal hyperexcitability is associated with epilepsy. To characterize neurophysiological abnormalities of the presently disclosed neuronal cell lines, intrinsic excitability of the neuronal cell lines is measured. In certain embodiments, the intrinsic excitability is measured by a whole-cell current-clamp recording device. Neuronal excitability can be assessed by several measures, e.g., the number of action potentials elicited by increasing current steps, the instantaneous firing frequency within a current step, action potential threshold (the membrane potential at which an action potential is generated; assessed separately with brief 1 ms current pulses that do not overlap with the action potential), and Rheobase (the smallest current necessary to trigger an action potential; assessed separately using a ramp current injection).

mTOR inhibitors can rescue neurite outgrowth defect and/or cell motility defect of the presently disclosed PMSE-derived neuronal cell lines. Additionally, mTOR inhibitors can reverse hyperexcitability, i.e., reduce intrinsic excitability, of the presently disclosed PMSE-derived neuronal cell lines. Therefore, the presently disclosed PMSE-derived neuronal cell lines can be used in an assay for identifying or screening an mTOR inhibitor. Such assay can include measuring the neurite outgrowth, the cell motility, and/or the intrinsic excitability of the presently disclosed PMSE-derived neuronal cell lines. The presently disclosed subject matter provides assays for identifying mTOR inhibitors, which assays include providing a neuronal cell line derived from a cell, e.g., a fibroblast, isolated from a subject diagnosed with PMSE, measuring initial neurite outgrowth of the neuronal cell line, contacting the neuronal cell line with a test compound and measuring resulting neurite outgrowth of the neuronal cell line contacted with the test compound. The reduction in phosphorylation of known mTOR substrates such as phospho-ribosomal S6 protein indicates that the test compound is an mTOR inhibitor. The neuronal cell lines can be contacted with the test compound for from about 3 days to about 7 days.

The presently disclosed subject matter further provides assays for identifying mTOR inhibitors, which assays include providing a neuronal cell line derived from a cell, e.g., a fibroblast, isolated from a subject diagnosed with PMSE, measuring initial cell motility of the neuronal cell line, contacting the neuronal cell line with a test compound and measuring resulting cell motility of the neuronal cell line contacted with the test compound. The reduction in phosphorylation of known mTOR substrates such as phospho-ribosomal S6 protein indicates that the test compound is an mTOR inhibitor. The neuronal cell lines can be contacted with the test compound for about 1 hour.

Furthermore, the presently disclosed subject matter provides assays for identifying an mTOR inhibitor, which assays include providing a neuronal cell line derived from a cell, e.g., a fibroblast, isolated from a subject diagnosed with PMSE, determining initial intrinsic excitability of the neuronal cell line, contacting the neuronal cell line with a test compound and determining resulting intrinsic excitability of the neuronal cell line contacted with the test compound. A reduction in the resulting intrinsic excitability in comparison to the initial intrinsic excitability indicates that the test compound is an mTOR inhibitor. The intrinsic excitability can be determined by measuring the intrinsic excitability with a whole-cell current-clamp recording device. The neuronal cell lines can be contacted with the test compound for from about 3 days to about 7 days.

5.4. Applications to Specific Diseases/Disorders

The PMSE-derived neuronal cell lines of the presently disclosed subject matter can be used in therapeutic methods for mTOR-associated diseases or disorders (Galanopoulou, 2012). mTOR pathway plays an essential role in cell growth, differentiation, proliferation and metabolism via phosphorylation of a number of translational regulators such as ribosomal S6 kinase and initiation factor 4E binding protein 1 (4EBP1) (Inoki et al. 2005; Crino 2011). In turn, mTOR pathway receives key information from nutrients, growth factors, cytokines, and hormones through tyrosine kinase receptors (Kwiatkowski 2003; Inoki et al. 2005). In brain cells, mTOR is also modulated by glutamate and dopamine receptors (Hoeffer et al. 2010). mTOR pathway can play a pivotal role during development of the cerebral cortex (Crino 2011). mTOR pathway is negatively regulated by tumor suppressor genes TSC1 and TSC2, as well as by their upstream regulators including phosphatase and tensin homolog (PTEN), STRADA and neurofibromin 1 (NF1) (Sulis et al. 2003; Puffenberger et al. 2007; Crino 2011; Ehninger et al. 2011). Mutations in these genes lead to hyperactivity of the mTOR pathway associated with cellular alterations including abnormal differentiation, proliferation and growth. mTOR dysregulation has been implicated in several genetic and acquired forms of epileptogenesis. Hyperactivity of mTOR pathway has been evidenced in a number of hypertrophic disorders of the brain including tuberous sclerosis complex (TSC) and Cowden disease (Inoki et al. 2005). Dysregulation of mTOR pathway can also be a common theme in focal cortical dysplasia (FCD), hemimegalencephaly and TSC. FCD is the most common cause of epilepsy in pediatric surgical cases (Lerner et al. 2009).

In certain embodiments, the mTOR-associated disease is epilepsy. There are at least eight neurodevelopmental epilepsy syndromes that are associated with altered mTOR activation, which include PMSE, tuberous sclerosis complex (TSC), hemimegelancephaly, focal cortical dysplasia, ganglioglioma, autism-macrocephaly syndrome (AMS), Fragile X syndrome (FXS), megalencephaly-polymicrogyria-polydactyly-hydrocephalus syndrome and megalencephaly-capillary malformation syndrome. mTOR inhibitors can reverse epileptogenic processes, and thus, have anti-epileptogenic effects. The anti-epileptogenic effects of mTOR inhibitors may depend upon the timing and dose of administration as well as the model used.

In certain embodiments, the PMSE-derived neuronal cell lines of the presently disclosed subject matter can be used in an assay for identifying or assaying anti-epileptogenic compounds. Such assays can include measuring the neurite outgrowth, the cell motility, or the intrinsic excitability of the presently disclosed PMSE-derived neuronal cell lines. The presently disclosed subject matter provides assays for identifying anti-epileptogenic compounds, which assays include providing a neuronal cell line derived from a cell, e.g., a fibroblast, isolated from a subject diagnosed with PMSE, measuring initial neurite outgrowth of the neuronal cell line, contacting the neuronal cell line with a test compound, and measuring resulting neurite outgrowth of the neuronal cell line contacted with the test compound. The ability of the test compound to reduce spontaneous action potentials or abnormal spiking indicates that the test compound is an anti-epileptogenic compound. Spontaneous action potentials or abnormal spiking can be detected by any known electrophysiological techniques, e.g., a patch clamp recording. The neuronal cell lines can be contacted with the test compound for from about 3 days to about 7 days.

The presently disclosed subject matter further provides assays for identifying anti-epileptogenic compounds, which assays include providing a neuronal cell line derived from a cell, e.g., a fibroblast, isolated from a subject diagnosed with PMSE, measuring initial cell motility of the neuronal cell line, contacting the neuronal cell line with a test compound and measuring resulting cell motility of the neuronal cell line contacted with the test compound. The ability of the test compound to reduce spontaneous action potentials or abnormal spiking indicates that the test compound is an anti-epileptogenic compound. Spontaneous action potentials or abnormal spiking can be detected by any known electrophysiological techniques, e.g., a patch clamp recording. The neuronal cell lines can be contacted with the test compound for about 1 hour.

Furthermore, the presently disclosed subject matter provides assays for identifying anti-epileptogenic compounds, which assays include providing a neuronal cell line derived from a cell, e.g., a fibroblast isolated from a subject diagnosed with PMSE, determining initial intrinsic excitability of the neuronal cell line, contacting the neuronal cell line with a test compound and determining resulting intrinsic excitability of the neuronal cell line contacted with the test compound. A reduction in the resulting intrinsic excitability in comparison to the initial intrinsic excitability indicates that the test compound is an anti-epileptogenic compound. The intrinsic excitability can be determined by measuring the intrinsic excitability with a whole-cell current-clamp recording device. The neuronal cell lines can be contacted with the test compound for from about 3 days to about 7 days.

Additionally, since PMSE patients exhibit neurobehavioral and cognitive deficits, the PMSE-derived neuronal cell lines of the presently disclosed subject matter can be used in an assay for screening or identifying compounds for autism and cognitive disability. The cells can be used to study the changes in dendritic structure, axon outgrowth, cell size, and electrical activity, all of which can be used as indices of network function in the intact brain. Abnormalities in these metrics have been identified in autistic brain tissue.

There are evolving areas in which mTOR pathway has been implicated such as spinal cord injury (Lu et al., 2012), hypoxia-ischemia (Chen et al., 2012), and Alzheimer's disease (Gouras, 2013) in which the PMSE-derived neuronal cell lines of the presently disclosed subject matter that exhibits constitutive mTOR activation can be used for assaying drug effects on for example, neurite outgrowth and cell signaling.

### 6. EXAMPLES

The following examples are merely illustrative of the presently disclosed subject matter and they should not be considered as limiting the scope of the presently disclosed subject matter in any way.

6.1. Generation of Fibroblast Derived iPSCs and Neurons

PMSE patient, parent, and normal control fibroblasts are obtained from skin-punch biopsies. Fibroblasts are extracted from tissue samples as described recently (Parker et al., 2013). Dermal fibroblasts are cultured as described in Liu et al, in press.

For reprogramming, 3×10⁵ cells in single cell suspension are electroporated with episomal plasmid DNA (1 μg each) for Sox2/Klf4, Oct3/4, 1-Myc and GFP using a Neon Electroporator and plated in iPSC medium (DMEM/F12, 20% knock-out serum replacement, 1 mM L-glutamine, 0.1 mM non-essential aminoacids, 100 U/ml pen/strep, 4 ng/ml of basic fibroblast growth factor [βFGF], 0.1 mM β-mercapto-ethanol). After a week, cells are plated onto mouse embryonic fibroblasts (MEFs) feeders and iPSC colonies picked manually 2-3 weeks later and plated on MEFs with weekly passage.

iPSC lines are characterized for expression of pluripotency genes, differentiation into three germ layers, and karyotype as described in Liu et al., in press. iPSC colonies are differentiated into neurons using a recently published dual SMAD inhibitor protocol (Shi et al., 2012). Briefly, iPSCs are plated on CellSTART-coated dishes with MEF-conditioned iPSC media, then switched to 3N medium with TGF-β inhibitors. After 8-10 days, cultures are passaged onto laminin-coated dishes. When neural rosettes form, the 3N medium is supplemented with 20 ng/ml βFGF (4 days). Rosettes are passaged onto Matrigel-coated dishes and incubated with 3N medium until the appearance of neurons around the rosettes, then dissociated into a single cell suspension and plated on a monolayer of rat astrocytes. After 48 hours, dividing neural progenitors are transduced with a retroviral vector containing mCherry driven by a human synapsin-1 promoter (RV-hSyn1-mCh) and placed back on astrocyte feeders.

Neurons and neural progenitors are fixed and stained using the same protocol as described for iPSCs. Primary antibodies are against: guinea pig anti-VGlut1 (1:500), rabbit anti-GABA (1:1000), mouse anti-Map2 a, b (1:500), mouse anti-βIII tubulin (1:4000), rabbit anti-GFAP (1:1000). For quality control, PMSE neurons are shown to be free of pathogens by in vitro assays for mycoplasma, HIV, Hepatitis B/C.

PMSE-derived neurons exhibit the identical genotype (STRADA deletion) identified in PMSE lymphoblasts and fibroblasts. Once generated, PMSE iPSCs are stored in liquid nitrogen for future use.

6.2. mTOR Activation in PMSE Neurons

The mTOR cascade/pathway is demonstrated to be activated in PMSE-derived neurons and in neural progenitor cells prior to differentiation (at the time the FGF is withdrawn from the media to initiate differentiation) to define at what point the mTOR cascade/pathway is activated. Protein lysates of PMSE-derived neurons are assayed by Western blotting (GAPDH as loading control) to define the relative phosphorylation levels of key mTOR regulatory proteins.

Assay of Altered Morphology: Neurite Length/Outgrowth.

Neurite length is measured by a blinded investigator in cells probed with β-III tubulin antibodies mounted on microscope slides (Fluoromount-G mounting media) for digital images to be captured (Leica DM4000 microscope and DFC340 FX camera; Fiji software package) and compared statistically (Student's t-test, p<0.05).

Cell Motility.

A modified version of the wound healing “scratch assay” developed in fibroblasts is used to quantify aberrant motility in PMSE neurons (Parker et al., 2013). Two dishes of neurons and neural progenitors, without rat astrocytes, per condition are grown until the cells reach confluence. Migration of PMSE neurons are determined in chamber slides, within a micro-incubator (model CSMI; Harvard Apparatus; at 37° C.) on an inverted microscope (Nikon TE300) equipped with a digital video camera (Evolution QEi; Media Cybernetics). Prior to the migration assay, cells are cultured for 24 hours in serum-deplete media to attenuate basal mTORC1 activity and then maintained for the duration of the migration assay. The surface of the dish is scratched with a P200 pipet tip creating a gap for cells to fill in and defining “time 0”. Images are taken every 5 minutes in the phase-contrast channel for 20 hours to follow the directional course of the cell front as well as individual cells. Migration differences are compared between the patient and control groups using independent-sample t-tests.

6.3. Effects of mTOR Inhibitors

For each of the above assays, PMSE-derived neurons are treated with one of mTOR inhibitors, targeting specific signaling nodes of the mTOR pathway: rapamycin (50-100 nM), a novel selective inhibitor of p70S6kinase1 (PF4708671; courtesy Pfizer; 10 μM), epigallocatechin gallate (EGCG), a major component of green tea that that potently inhibits both PI3K and mTOR, Torin1 (a dual mTORC1 and mTORC2 inhibitor), BEZ235 (a dual mTOR-PI3K inhibitor), BKM120 (a pan-PI3K inhibitor), or BYL719 (a PI3Kalpha inhibitor; all courtesy of Novartis; effective concentrations to be determined), or metformin an inhibitor of AMPK to determine an effect on mTOR signaling. As a drug treatment control, cells are treated with DMSO. Each compound is tested on neural progenitor cells for 3, 5, and 7 days or on fully differentiated PMSE neurons to show that blockade of mTOR can occur at both stages.

In the neurite outgrowth experiments, PMSE-derived neurons are treated with mTOR inhibitors for 3, 5, and 7 beginning on the day of final plating (a time when neurites are first extended). Mean neurite length is measured in digital images as above and compared statistically (Student's t-test, p<0.05) across each treatment condition by a blinded investigator.

In the motility assay, mTOR inhibitors are applied for one hour prior to and throughout the duration of the experiment (optimal doses to be determined) to show that motility defect in PMSE neurons can be rescued by mTOR pathway inhibition.

It is shown that transfection of PMSE-derived neurons with a full length human STRADA plasmid-GFP plasmid construct (Addgene) (available by using Lipofectamine Plus reagent) can rescue the signaling, neurite outgrowth, and motility defects. At 3, 5, and 7 days post-transfection, GFP expressing cells are FAC sorted at SHPRC and protein lysates generated to assay normalization of mTOR signaling as a consequence of normal STRADA expression.

6.4. Electrophysiology

The identification of functional deficits in patient-derived human neurons offers a tremendous opportunity for identifying novel therapeutics. For this reason, the neurophysiological abnormalities in PMSE-derived neurons were characterized. Synaptic transmission and plasticity in these PMSE-derived neurons can be fully characterized. First intrinsic neuron excitability of these PMSE-derived neurons was characterized because of the strong logical connection between neural hyperexcitability and epilepsy. The results showed that PMSE-derived neurons recapitulated the hyperactive mTORC1 signaling seen in patients and exhibited a striking increase in intrinsic neuron excitability relative to control neurons. Since altered neural excitability is likely to disrupt information processing in neural circuits and can predispose neurons to synchronous activation, this phenotype provides a strong candidate cellular mechanism underlying both the intellectual disability and epilepsy that are pervasive in PMSE, and other disorders associated with hyperactive mTORC1 signaling.

6.5. Characterization of Neurophysiological Abnormalities in PMSE Neurons.

A full characterization of the altered intrinsic excitability in PMSE-derived neurons is conducted to assess action potential properties to gain insight into the molecular mechanisms underlying this PMSE phenotype. It is focused first on excitatory neurons. To measure intrinsic excitability, whole-cell current-clamp recordings are made with an Axopatch 200B, Multiclamp 700, or Axopatch1-D amplifier from PMSE and control neurons bathed in HEPES-buffered saline (HBS; containing, in mM: 119 NaCl, 5 KCl, 2 CaCl2, 2 MgCl2, 30 Glucose, 10 HEPES, pH 7.4) plus 10 μM CNQX, 20 μM APV and 10 bicuculline to remove background synaptic input by blocking glutamate and GABA receptor activation. Recording electrodes are filled with an internal solution containing, in mM: 115 KMeSO4, 15 KCl, 5 NaCl, 0.02 EGTA, 1 MgCl2, 10 Na2-Phosphocreatine, 4 ATP-Mg, 0.3 GTP-Na, pH 7.2, and have resistances ranging from 5-7 MΩ. Series resistance is compensated by >90%. Input resistance, resting membrane potential, and capacitance are monitored throughout experiments to ensure high quality recordings. Intrinsic excitability is assessed by using a series of 1000-ms step current injections increasing in 5 pA increments delivered every 10-s.

Neuronal or neural excitability is assessed by several measures: the number of action potentials elicited by increasing current steps, the instantaneous firing frequency within a current step, action potential threshold (the membrane potential at which an action potential is generated; assessed separately with brief 1 ms current pulses that do not overlap with the action potential), and Rheobase (the smallest current necessary to trigger an action potential; assessed separately using a ramp current injection). Action potential waveforms of a single action potentials will be defined using 1 ms current steps to elicit single spikes, as well as the waveform following a train of 5 spikes elicited at 20 Hz (to assess spike afterhyperpolarization; AHP). Action potential properties and intrinsic excitability data are analyzed using Clampfit (Molecular Devices; ANOVA and Tukey-Kramer post-hoc test).

6.6. Testing mTOR Inhibitors in Reversing Hyperexcitability in PMSE-Derived Neurons

PMSE-derived neurons are treated for 3, 5, or 7 days with mTOR inhibitors starting 1 week after plating and their intrinsic excitability and AP waveforms assessed as described above. Drugs are administered in media, and refreshed every 48 hours during media exchanges to ensure continuity of action. Appropriate concentrations are established and the effectiveness of these treatment regimens for reversing hyperactive mTORC1 signaling are verified in Aim 1. A “corrected” or rescued phenotype is defined as a reduction in intrinsic excitability in PMSE-derived neurons to control levels while having no effect on intrinsic excitability in control neurons over the same course or treatment. The hyperexcitability observed in PMSE-derived neurons provides an exciting phenotype to screen novel therapeutics that target mTOR signaling at different pathway nodes.

The analysis of the effects of mTOR inhibitor on pre- and post-differentiation PMSE-derived neurons permits a developmental assay for how aberrant morphology and electrophysiology can be both rescued and prevented. It is focused first on excitatory neurons. GABAergic interneurons or astrocytes can be selectively differentiated. A thorough characterization of excitability changes and spike waveforms in PMSE-derived neurons will reveal specific ion channels likely to be aberrantly regulated. For example, changes in rheobase and action potential threshold are consistent with altered expression of voltage-gated Na+ channels, whereas in mice, mTORC1 has been shown to inhibit the translation of the low-threshold K+ channel Kv1.1 (Raab-Graham et al., 2006), so diminished Kv1.1 current could also contribute. Other changes (e.g., AHP) could reflect altered Ca2⁺-activated K⁺ channels which can be further investigated. Expression of neural Na⁺ channel alpha subunits (NaV1.1, NaV1.2, and Nav1.6), Kv1.1, or Ca2⁺-activated K⁺ channels is analyzed by Western assay or immunocytochemistry. Together, these experiments provide a deeper understanding of the mechanisms underlying hyperexcitability in PMSE-derived neurons, which will provide specific molecular targets to add for screening of potential therapeutic agents. In view of the effects of sirolimus on PMSE patients, mTOR inhibitors can effectively reverse the hyperactivity phenotype of PMSE neurons. If treatment with an mTOR inhibitor corrects the hyperactivity phenotype, it is examined whether this reverses when treatment is discontinued, and if so, the timing of that effect is examined, e.g., examine 3, 5, and 7 days following treatment cessation. The effects of acute administration (60 min) of successful compounds are also examined to rule out a potential direct effect on ion channels that could account for the reduced excitability. An alternative possibility is that multiple compounds will be partially effective in each assay, but lack the ability to fully restore normal neurite outgrowth, motility, or excitability in PMSE neurons. In cases where a partial rescue is observed, combined treatment of multiple agents are explored to see if a more complete rescue is possible.

Although the presently disclosed subject matter and its advantages have been described in detail, it should be understood that various changes, substitutions and alterations can be made herein without departing from the spirit and scope of the presently disclosed subject matter as defined by the appended claims. Moreover, the scope of the presently disclosed subject matter is not intended to be limited to the particular embodiments described in the specification. Accordingly, the appended claims are intended to include within their scope such modifications.

Patents, patent applications, publications, product descriptions, and protocols that may be cited throughout this application, the disclosures of which are incorporated herein by reference in their entireties for all purposes.

What is claimed is:

1. An induced pluripotent stem cell-derived neuronal cell line from a subject diagnosed with polyhydramnios-megalencephaly-symptomatic-epilepsy (PMSE).

2. The neuronal cell line of claim 1, wherein PMSE results from a recessive mutation in STE20-related adaptor protein alpha (STRADA) gene.

3. The neuronal cell line of claim 2, wherein the recessive mutation is a homozygous deletion of exons 9-13 of the STRADA gene.

4. The neuronal cell line of claim 1, wherein the neuron cell line exhibits increased or decreased mammalian target of rapamycin (mTOR) signalling.

5. The neuronal cell line of claim 4, wherein the neuron cell line exhibits increased mTOR signalling.

6. The neuronal cell line of claim 1, wherein the neuron cell line exhibits neurite outgrowth defect.

7. The neuronal cell line of claim 1, wherein the neuron cell line exhibits cell motility defect.

8. The neuronal cell line of claim 1, wherein the neuron cell line exhibits neuronal hyperexcitability.

9. The neuronal cell line of claim 8, wherein neuronal hyperexcitability is associated with epilepsy.

10. An assay for identifying an mTOR signalling modulator, comprising:
(a) providing an iPSC-derived neuronal cell line from a subject diagnosed with PMSE;
(b) determining or measuring initial intrinsic excitability of the neuronal cell line, initial neurite outgrowth of the neuronal cell line, initial cell motility of the neuronal cell line, and/or initial phosphorylation status of at least one mTOR substrate of the neuronal cell line;
(c) contacting the neuronal cell line with a test compound; and
(d) determining resulting intrinsic excitability of the neuronal cell line contacted with the test compound, resulting neurite outgrowth of the neuronal cell line contacted with the test compound, resulting cell motility of the neuronal cell line contacted with the test compound, and/or resulting phosphorylation status of the at least one mTOR substrate of the neuronal cell line contacted with the test compound;
wherein at least one of the followings indicates that the test compound is an mTOR signalling modulator:
(i) a change in the intrinsic excitability in the presence of the test compound in comparison to the intrinsic excitability in the absence of the test compound;
(ii) a change in the neurite outgrowth in the presence of the test compound in comparison to the neurite outgrowth in the absence of the test compound;
(iii) a change in the cell motility in the presence of the test compound in comparison to the cell motility in the absence of the test compound; and
(iv) a reduction in the phosphorylation status of the at least one mTOR substrate in the presence of the test compound in comparison to the phosphorylation status of the at least one mTOR substrate in the absence of the test compound.

11. The assay of claim 10, wherein the intrinsic excitability is determined by measuring the intrinsic excitability with a whole-cell current-clamp recording device.

12. The assay of claim 10, wherein the neuronal cell line is contacted with the test compound for from about 3 days to about 7 days.

13. The assay of claim 10, wherein the neuronal cell line is contacted with the test compound for about 1 hour.

14. The assay of claim 10, wherein the at least mTOR substrate is selected from the group consisting of ribosomal S6 protein, death-associated protein 1 (DAP1), and Autophagy-related protein 13 (ATG13),

15. The assay of claim 10, wherein measuring the phosphorylation status is by a method selected from the group consisting of Western blot and immunohistochemistry.

16. An assay for identifying an anti-epileptogenic compound, comprising:
(a) providing an iPSC-derived neuronal cell line from a subject diagnosed with PMSE;
(b) determining or measuring initial intrinsic excitability of the neuronal cell line, initial neurite outgrowth of the neuronal cell line, initial cell motility of the neuronal cell line, initial phosphorylation status of at least one mTOR substrate of the neuronal cell line, initial spontaneous action potentials of the neuronal cell line, and/or initial abnormal spiking of the neuronal cell line;
(c) contacting the neuronal cell line with a test compound; and
(d) determining resulting intrinsic excitability of the neuronal cell line contacted with the test compound, resulting neurite outgrowth of the neuronal cell line contacted with the test compound, resulting cell motility of the neuronal cell line contacted with the test compound, resulting phosphorylation status of the at least one mTOR substrate of the neuronal cell line contacted with the test compound, resulting spontaneous action potentials of the neuronal cell line contacted with the test compound, and/or resulting abnormal spiking of the neuronal cell line contacted with the test compound,
wherein at least one of the followings a indicates that the test compound is an anti-epileptogenic compound:
(i) reduction in the intrinsic excitability in the presence of the test compound in comparison to the intrinsic excitability in the absence of the test compound;
(ii) a reduction in the neurite outgrowth in the presence of the test compound in comparison to the neurite outgrowth in the absence of the test compound;
(iii) a reduction in the cell motility in the presence of the test compound in comparison to cell motility in the absence of the test compound;
(iv) a reduction in the phosphorylation status of the at least one mTOR substrate in the presence of the test compound in comparison to the phosphorylation status of the at least one mTOR substrate in the absence of the test compound;
(v) a reduction in the spontaneous action potentials in the presence of the test compound in comparison to the spontaneous action potentials in the absence of the test compound; and
(vi) a reduction in the abnormal spiking in the presence of the test compound in comparison to the abnormal spiking in the absence of the test compound.

17. The assay of claim 16, wherein the intrinsic excitability is determined by measuring the intrinsic excitability with a whole-cell current-clamp recording device.

18. The assay of claim 16, wherein the neuronal cell line is contacted with the test compound for from about 3 days to about 7 days.

19. The assay of claim 16, wherein the neuronal cell line is contacted with the test compound for about 1 hour.

20. The assay of claim 16, wherein the at least mTOR substrate is selected from the group consisting of ribosomal S6 protein, death-associated protein 1 (DAP1), and Autophagy-related protein 13 (ATG13).

21. The assay of claim 16, wherein measuring the initial and resulting phosphorylation status is by a method selected from the group consisting of Western blot and immunohistochemistry.

22. The assay of claim 16, wherein measuring the spontaneous action potentials is by an electrophysiological technique.

23. The assay of claim 22, wherein the electrophysiological technique is a patch clamp recording.

24. The assay of claim 16, wherein measuring the spiking is by an electrophysiological technique.

25. The assay of claim 24, wherein the electrophysiological technique is a patch clamp recording.
